(19) United States
(12) Patent Application Publication
(10) Pub. No.: US 2013/0273583 A1
(43) Pub. Date: Oct. 17, 2013

(54) TESTING APPARATUS FOR DETECTION OF GASTROINTESTINAL DISORDERS

(71) Applicant: Thomas Julius Borody, Sidney (AU)

(72) Inventor: Thomas Julius Borody, Sidney (AU)

(73) Assignee: CENTRE FOR DIGESTIVE DISEASES PTY LIMITED, Five Dock (AU)

(21) Appl. No.: 13/880,295

(22) PCT Filed: Oct. 17, 2011
(86) PCT No.: PCT/AU2011/001320

§ 371 Date: Jun. 5, 2013

(30) Foreign Application Priority Data
Oct. 18, 2010 (AU) 2010904631

(51) Int. Cl. C12Q 1/58

(52) U.S. Cl. CPC C12Q 1/58
USPC 435/12

(57) ABSTRACT

Detection of urease in gastric samples is achieved by using a composition comprising urea and an indicator. This composition can be provided in the form of a liquid or in a number of dry formats including a wafer, impregnated material, coated well or a tablet. The assay can be performed in described apparatus comprising a well which can be provided with a removable air-tight seal. In one optional form, the well may have a swaged rim for retaining a disc or ring inside the well and hinged plugs. This assay is useful for detecting H. pylori related gastrointestinal conditions

### TECHNICAL FIELD

The present invention relates to a composition and diagnostic device or testing apparatus suitable for use in diagnosing disorders in humans or other mammals. In particular, the present invention is directed towards a composition and testing apparatus for diagnosing gastrointestinal disorders by detecting the presence of the enzyme called urease.

### BACKGROUND

Helicobacter pylori is a bacterium which can infect the gastric mucosal surface and can cause acute and chronic histological gastritis, duodenal ulcer, gastric ulcer, non-ulcer dyspepsia, and possibly gastric cancer and MALT lymphoma. The presence of Helicobacter pylori can be diagnosed by a blood test (serology), non-invasively by a Urea Breath Test, or by endoscopic tests where an endoscope is inserted in the stomach, a piece of the mucosa is collected by biopsy forceps and ether culture, histological examination or rapid urease test carried out to detect for the presence of the bacteria. The reason why a rapid urease test can be carried out is that Helicobacter pylori produces vast amounts of the enzyme called ‘urease’ which can be detected by special ‘kit of parts’ containing, among other components, urea and an indicator. Upon detecting the presence of H. pylori patients can then be treated with combinations of antibiotics to cure the infection and thereby treat an ulcer, non-ulcer dyspepsia or MALT lymphoma, when present.

As with the management of any disease, a rapid, precise and accurate diagnostic agent for the condition is of paramount importance. However the diagnostic methods currently available such as histopathology and culture are cumbersome, slow, as well as costly. Although Helicobacter pylori can be detected by serology, the tests can remain positive for many months after the bacteria have been eradicated so that checking for success of eradication is also not reliable.

Other methods of detecting H. pylori, including the C13 and C14 breath tests, require the availability of expensive isotopes, detection machines and expertise to carry out the test.

It is known that Helicobacter pylori can be detected by the presence of its urease enzyme in so the gastric mucosa or in the gastric fluid of humans. U.S. Pat. No. 4,748,113 (Marshal) describes a test kit for urease enzyme detection (CLO-Test). However this test kit requires storage at 2-8° C. because it contains a wet component consisting of agar and the agar progressively dries out giving the CLO-Test a relatively short shelf life. Furthermore, some nurses encounter difficulty in actually inserting the gastric biopsy fragment into the agar well in the CLO-Test. A further test method, described as Pyloritek and claimed in U.S. Pat. Nos. 5,314,804 and 5,420,016, utilizes a number of components such as a test part incorporating a diffusion element reaction chamber and hydrating solution. This test kit is also cumbersome, requires storage at 2-7° C. and can be messy to use.

Borody and Shortis in U.S. Pat. No. 6,649,360 describes a dry test where the urea-containing solution is dried within a specialised blotting paper allowing the gastric tissue to react directly with the urease product dried in the blotting material. A similar product. Pronto Dry suffers from the problem of an early blush of purple resulting in false-positive responses upon insertion of the biopsy. A more recent patent, U.S. Pat. No. 6,929,926 (Marshal) describes a two-component powdered system for detection of H. pylori. However, going through the description of the process of carrying out the test, the biopsy needs to be contacted with the powder in two steps and it runs the risk of lack of reaction due to the relative lack of water in the biopsy tissue. It is certainly cumbersome in its description.

Hence, the limitations of the existing tests are clinically real, particularly in environments where storage and shipping are an issue. Helicobacter pylori detection is also relevant in developing countries where the bulk of human Helicobacter pylori infection is now found. However the cost of the present tests is too high to be used in these counties and so cost reduction is an issue.

An object of this invention is to solve the problems of high cost and complexity of the urease test or at least provide a suitable alternative.

### DEFINITIONS

The following are some definitions that may be helpful in understanding the description of the present invention. These are intended as general definitions and should in no way limit the scope of the present invention to those terms alone, but are put forth for a better understanding of the flowing description.

Unless the context requires otherwise or specifically stated to the contrary, integers, steps, or elements of the invention recited herein as singular integers, steps or elements clearly encompass both singular and plural forms of the recited integers, steps or elements.

Throughout this specification, unless the context requires otherwise, the word “comprise”, or variations such as “comprises” or “comprising”, will be understood to imply the inclusion of a stated step or element or integer or group of steps or elements or integers, but not the exclusion of any other step or element or integer or group of elements or integers. Thus, in the context of this specification, the term “comprising” means “including principally, but not necessarily solely”.

The information provided herein and references cited are provided solely to assist the understanding of the reader, and do not constitute an admission that any of the references or information is prior art to the present invention.

The term “tablet” means a small, solid form of the composition, but does not include a powder or gel. It may be compressed or molded in its manufacture, and it may be of any size, shape including round, oval, rectangular, square, ring or cylinder, weight, and colour but is most desirably of a rounded form such as a small disc or cylinder. It may also be in the form of a ring. Typical thicknesses range from 0.1 mm to 1.5 cm. Typical diameters range from 0.2 to 5 cm.

By “wafer” is meant a flat, solid form of the composition, suitably a very thin tablet. The wafer may be in any suitable shape, including round, oval, rectangular, or square. Typical thicknesses range from 0.01 to 2 mm.

By “coated” is meant a portion or layer of the dry indicating composition covers an underlying to material. By “impregnated” is meant the dry indicating composition permeates, is partially absorbed by or is fully saturated by a material.

By “material” is meant any material capable of supporting or absorbing the dry indicating composition. Suitable materials include films and paper. The material may be cut into suitable shapes such as described above, but preferably cut into discs or rings.

The term “well” or “well system” means any receptacle capable of receiving and containing a material. Suitable webs include a spoon, test tube, or other type of well system such as described in the accompanying Figures.

By “drying” the indicating composition means that a liquid composition is allowed to dry at a temperature, which can be of the order of room temperature up to 80° C. which is warm enough to dry the liquid composition, suitably in a short amount of time, yet cool enough that the underlying material is not affected.

As used herein, ‘gastrointestinal disorder’ encompasses any disease or other disorder of the gastrointestinal tract of a human or lower mammal. Such gastrointestinal disorders include, for example: disorders not manifested by presence of ulcerations in the gastric mucosa (herein “no ulcerative gastrointestinal disorder”), including chronic or atrophic gastritis, gastroenteritis, non-ulcer dyspepsia, oesophageal reflux disease and gastric motility disorder, and “peptic ulcer disease” i.e, gastric and duodenal ulcers. In particular, “gastrointestinal disorder” refers to such disorders of the upper gastrointestinal tract caused or mediated by bacteria, including Helicobacter-like organisms, e.g, Helicobacter pylori. Such Helicobacter-like organisms include those described J. R. Warren and B. J. Marshall, “Unidentified Curved Bacilli on Gastric Epithelium of Campylobacter-like Bacteria that are Distinct from Campylobacter jejuni”. The Lancet 111-112 (1985).

As used herein, “gastric material” refers to any moist material obtained directly or indirectly from the gastrointestinal tract of a human or other mammal. Such materials include, for example, gastric epithelium, gastric mucosa, gastric tissue and digestive fluids. Samples of such materials, for the use in the method of the invention, may be obtained by a variety of well known methods, according to sound medical practice. Such methods include, for example, obtaining the sample by biopsy of the subject, obtaining the sample from vomitus of the subject, and obtaining the sample from nasal gastric aspirate.

As used herein, “contacting” refers to any method which effects substantial interface between a dry indicating composition and the sample gastric material, optionally in the presence of water, for a time sufficiently long so as to allow the hydrolysis of urea by any urease present in the sample.

### DESCRIPTION OF DRAWINGS

FIG. 1 is a top view 1A and a cross-sectional view 1B of a well system for receiving a tablet or wafer in accordance with one embodiment of the present invention.

FIG. 2 are perspective views 2A, 2B and 2C, top view 2D and cross-sectional view 2E of a well system for receiving a coated or impregnated disc or ring in accordance with one embodiment of the present invention. FIG. 2F shows a cross-section of the well system during use.

FIG. 3 is a top view 3A and cross-sectional view 3B of a well system for containing a quid indicating composition in accordance with one embodiment of the present invention.

### SUMMARY OF THE INVENTION

In one aspect the invention provides a testing apparatus for detecting urease in a gastric material taken from a mammal, said testing apparatus in the form of a tablet, wafer, a coated or impregnated material or a coated well, the tablet, wafer, coated or impregnated material or coated well comprising a dry indicating composition responsive to urease, wherein the dry indicating composition comprises urea and an indicating agent.

In an embodiment the indicating agent is selected from the group consisting of phenol red, p-nitro-phenol, bromothymol blue, neutral red, quinoline blue, cresol red, metacresol purple, thymol blue, bromocresol purple, chlorophenol red, bromocresol green and bromophenol blue.

In an embodiment the dry indicating composition is prepared by drying a stock solution comprising, per 200 mL, urea 2 g to 10 g and an indicating agent.

In an embodiment of the tablet or wafer, the urea has a mean particle size of less than 0.01 mm.

In an embodiment the dry indicating composition further includes one or more of disintegrants, bacteriocidal agents preservatives, sodium dioxide, sodium alumino silicate, buffering agents, pH adjusting agents, solubilizing agents, wetting agents, diluents, fillers, binders, lubricants, colourants, stabilizers, surfactants and agar.

In an embodiment the material of the coated or impregnated material is filter paper or blotting paper.

In an embodiment the coated or impregnated material is in the form of a disc or ring.

In one aspect the invention provides a method of preparing a testing apparatus for detecting urease in a gastric material taken from a mammal, the testing apparatus in the form of a tablet, wafer, a coated or impregnated material or a coated well, the tablet, wafer, coated or impregnated material or coated well comprising a dry indicating composition responsive to urease, wherein the dry indicating composition comprises urea and an indicating agent, said method comprising compressing or molding urea and an indicating agent to form a tablet or a wafer.

In one aspect the invention provides a method of preparing a testing apparatus for detecting urease in a gastric material taken from a mammal, the testing apparatus in the form of a tablet, wafer, a coated or impregnated material or a coated well, the tablet, wafer, coated or impregnated material or coated well comprising a dry indicating composition responsive to urease, wherein the dry indicating composition comprises urea and an indicating agent, said method comprising immersing a material into a liquid comprising urea and an indicating agent, followed by drying the liquid so as to form a coated or impregnated material.

In an embodiment the method further comprises cutting the material into any suitable shape, such as a disc or ring.

In one aspect the invention provides a method of preparing a testing apparatus for detecting urease in a gastric material taken from a mammal, the testing apparatus in the form of a tablet, wafer, a coated or impregnated material or a coated well, the tablet, wafer, coated or impregnated material or coated well comprising a dry indicating composition responsive to urease, wherein the dry indicating composition comprises urea and an indicating agent, said method comprising coating a well with a liquid comprising urea and an indicating agent, followed by drying the liquid so a to form a coated well.

In one aspect the invention provides a testing apparatus for detecting urease in a gastric material taken from a mammal, said testing apparatus in the form of a well containing a liquid composition responsive to urease, wherein the liquid indicating composition comprises urea and an indicating agent and the well is provided with a removeable air-tight seal.

In one aspect the invention provides a method of preparing a testing apparatus for detecting urease in a gastric material taken from a mammal, said testing apparatus in the form of a well containing a liquid composition responsive to urease, wherein the liquid indicating composition comprises urea and an indicating agent and the well is provided with a removeable air-tight seal, the method comprising depositing into a well a liquid comprising urea and an indicating agent, followed by sealing the well with a removable air-tight seal.

In one aspect the invention provides a method for detection of a gastrointestinal disorder in a mammal by detection of urease in gastric material of the mammal, comprising the steps of: (a) providing the testing apparatus of the invention, (b) obtaining a sample of gastric material from said mammal; (c) contacting the indicating composition with the gastric sample, optionally in the presence of water; and (d) observing a colour change, wherein a change in colour indicates the presence of urease and the existence of a gastrointestinal disorder in the mammal.

In one embodiment the contacting occurs in a well in which the tablet, wafer, coated or impregnated material has been placed to which water is added so as to bring the dry indicating composition into solution.

In one embodiment the contacting occurs in the coated well to which water has been added to as to bring the dry indicating composition into solution.

In one aspect the invention provides a method for detection of a gastrointestinal disorder in a mammal by detection of urease in gastric material of the mammal, comprising the steps of:

(a) providing a testing apparatus in the form of a well containing a liquid composition responsive to urease, wherein the liquid indicating composition comprises urea and an indicating agent and the well is provided with a removeable air-tight seal,

(b) obtaining a sample of gastric material from said mammal,

(c) removing the air-tight seal and contacting the indicating composition with the gastric sample; and

(d) observing a colour change, wherein a change in colour indicates the presence of urease and the existence of a gastrointestinal disorder in the mammal.

In one embodiment of the method for detection of a gastrointestinal disorder in a mammal, the gastrointestinal disorder is a disorder associated with the presence of a Helicobacter-like organism. In one embodiment the Helicobacter-like organism is Helicobacter pylori.

In one aspect the invention provides a well system having a well and integrally hinged plug, wherein the well includes a swaged rim for retaining a disc or ring inside the well.

The present invention is presented in its several formats and each is a variation of a similar invented theme. It overcomes or ameliorates disadvantages associated with the original Marshall patent which had the urease test within an agar gel. It overcomes the disadvantages because the original test requires refrigeration of the kits and has the problem of the agar drying out and shriveling, so that it has a short refrigerator shelf life after which it has to be discarded, being dry and ineffective. U.S. Pat. No. 6,649,360 (Borody) reduces the cost, but incompletely, and the present invention overcomes the disadvantages of U.S. Pat. No. 6,649,360 on cost and simplicity.

The present invention is based on the known action of urease to convert urea into ammonium carbonate. This molecule then decomposes into ammonia and carbon dioxide. Consequently, a gastric material is suitably placed into contact with the composition of the present invention, which composition contains also contains, in addition to urea, an indicator, suitably a pH indicator which changes colour when there is a rise in the pH. If urease is present within the gastric material, it breaks down the urea which then results in the formation of ammonia, thereby elevating the pH and turning the test positive. The gastric material, suitably in form of a biopsy, may be collected endoscopically from the patient and contacted with at least one of the invented format(s) of the composition of the present invention. There a several novel formats or presentations of the composition. It is the way the chemical composition is presented to the gastric material that makes this test simple to use and cheap to bring to the patient and not complex or expensive. Several aspects of this invention deal with or describe the format or presentation of the chemical composition.

The composition of the invention may be used to test gastric material taken from a human or other animal. There is no need to refrigerate the composition, the composition can be stored at room temperature.

The first aspect of the present invention is that the chemical reacting composition (dry indicating composition) is in the form of a tablet. It is not a liquid, it is not a gel, it is not a powder as in previous inventions. It is in the form of a tablet which can be inserted into a well where the urease reaction then takes place. Its presence in the tablet format gives the test marked advantages previously not appreciated by other inventors. These advantages include the ability of the tablet to be placed into either a test tube, a simple prepared well, or any other well-like object even a small spoon (suitable for use for example in a third world country). Suitably, the addition of a small volume of water to the tablet into the ‘well’ then allows the tablet go into solution, thereby allowing the final reactive composition to be made up on the spot and a collected endoscopic biopsy (gastric material) may then be placed into the solution for testing. This is of particular importance in developing counties where the cost of urease testing would be prohibitive when using the currently accompanying manufactured plastic components.

The major advantages of the required urea mixture of reagents being in the form of a tablet include the fact that it is a quantum amount of the components, it is relatively heap, the pH does not shift or drift in the dry tablet (as it does in a stored liquid solution or in agar). It is also suitable of a standard size and it can be used in any type of well, either a cheap manufactured well system, test tube or even a spoon.

In one embodiment, the tablet will contain the following components: urea; and an indicator such as phenol red.

Other additional excipients may be included. For example a disintegrant, a bacteriocidal agent or preservative (for example sodium azide), sodium dioxide, sodium alumino silicate powder, buffering agents, solubilizing agents, wetting agents, diluents, filers, binders, lubricants, colourants, stabilizers and/or surfactants may be included.

A typical stock solution (200 ml) may contain the following components (suitable range provided in brackets):—

TABLE

| | | |
|---|---|---|
| Urea | 10 | g (2-50 g) |
| Phenol Red (0.5% w/v) | 50 | ml (2-100 mg/L) |
| NaH₂PO₄•H₂O | 0.218 | g (0.01-2.0 g) |
| NaHPO₄ | 0.51 | g (0.01-5.0 g) |
| NaN₃ | (sodium azide) 100 | mg (2-1000 mg) |

Dissolved in 200 ml Deionised water and brought to a pH of 6.85.

In one embodiment, the tablet is a compressed or molded powder of all of the ingredients.

In detail, the urea component, in particular, will suitably be of a fine grade to allow for the most rapid reaction with the Helicobacter pylori bacterial urease and the specific commercial grade will suitably have a urea ground to a mean particle size of <0.1 mm but even more so <0.05 mm or preferably <0.01 mm. The smaller the particle size, the faster the reaction as attaches itself better to the biopsy samples (gastric material) and individual bacterial cells. The urea content is suitably in the range given in the table above.

The indicators useful in this invention are weak acids, with sharply different colors in their dissociated (ionised) and undissociated (neutral) states. The indicator may be present at any effective concentration, being any concentration or amount that provides a discernable colour change when used according to the invention.

In one embodiment, the indicator may be phenol red but may also be p-nitro-phenol, bromothymol blue, neutral red, quinoline blue, acrd red, metacresol purple, thymol blue, bromocresol purple, chlorophenol red, bromocresol green or bromophenol blue. In one embodiment the indicator has a pKₐ of from 5 to about 8.5, suitably from about 6.5 to about 8, in the so final reactive composition. In one embodiment the indicator has a pKₐ in the range of from about 6.7 to about 6.85. In one embodiment the indicator has a pKₐ of about 6.85.

A pH adjusting agent may also be provided to maintain a pH inside the tablet of between 4 and 6.5 pH, for example a pH of 5 or 6, and may include sodium citrate, phosphate citrate buffer, citric acid, sulfamic acid, sodium bisulphate, sodium acetate, sodium phosphate and potassium phosphate. The bacteriocidal agent is preferably sodium azide or methyl paraben (methyl-p-hydroxybenzoate) or may be propyl paraben.

In one embodiment the pH of the composition is at least about one pH unit lower than the pKₐ of the indicator.

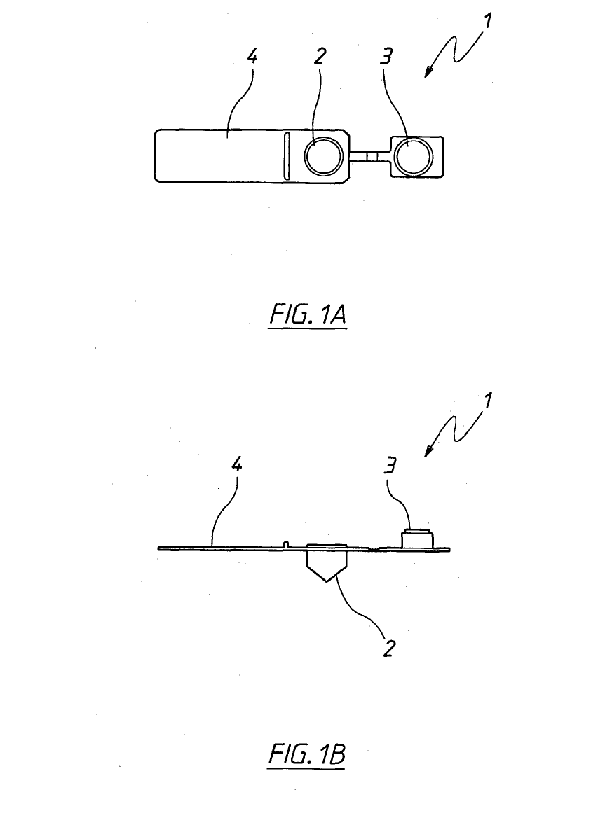

In one embodiment, the tablet containing the composition as given in the table above is inserted into either a test tube, placed on a spoon or inserted into a well system such as shown in FIG. 1. With reference to FIG. 1, the well system 1 shown in FIG. 1 or receiving a tablet in accordance with the present invention includes a solution well 2 to hold the tablet and an integral hinged plug 3 for replacing over the well once the tablet has been inserted.

The well system may be an individual well or more than one wed may be used, for example a well system may be made in strips of, for example, 10 wells, ready for an endoscopy session where more than one patient or gastric material sample is tested. In this embodiment, the tablets may be inserted into the bottom of each well and several drops of sterile water may be added, suitably at the beginning of the endoscopy list for the day. The tablets will then suitably dissolve and form a ready urease liquid for the day's endoscopy session. During endoscopy, a sample of gastric mucosa is suitably collected and inserted into the well and the reaction with turn either positive or remain negative for Helicobacter pylori, suitably turning from yellow to purple colour to indicate the presence of urease in the wall of the Helicobacter pylori cells.

In a second aspect, the tablet format of the invention can also be simplified or substituted. Instead of the tablet, in the second variation or aspect of the invention, the tablet can be substituted by a thin wafer (a very thin tablet, almost the thickness of a paper suitably made up of the same components listed in the above table) but able to dissolve much more rapidly. This wafer can also be inserted into any well system and used in the same manner as described for the tablet format of the invention.

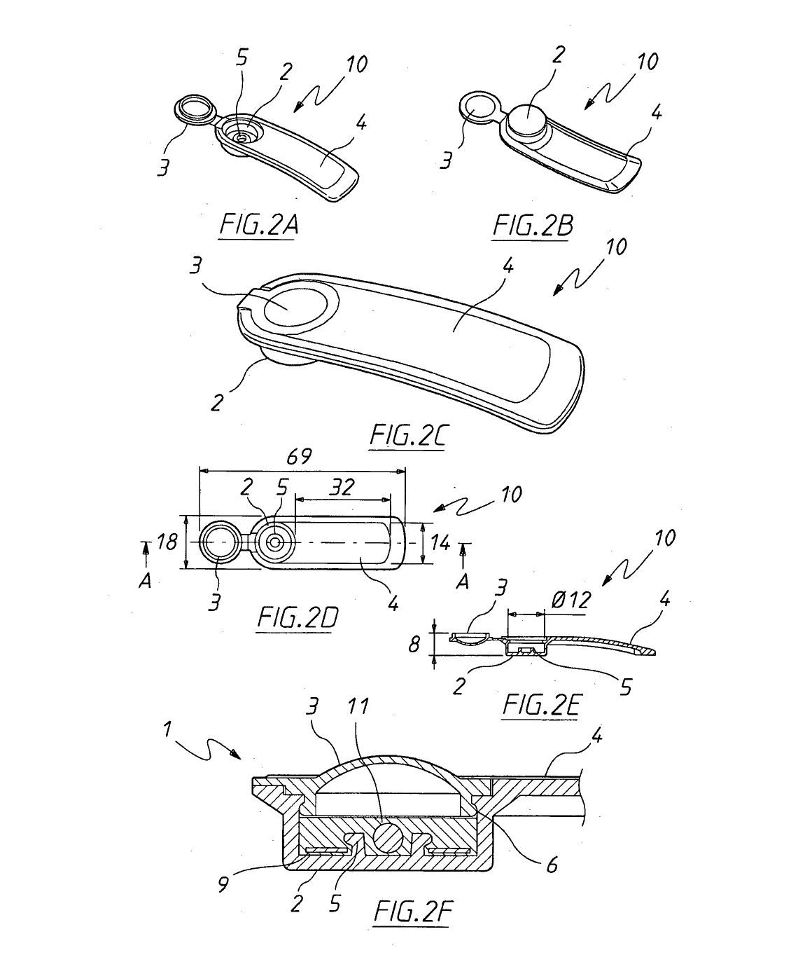

In the third aspect of the invention the tablet can be replaced in any well system by material, suitably a disc or ring (disc with general round hole) of a suitable material capable of carrying the dry indicating composition by coating, absorption or impregnation, for example filter or blotting paper or a film soaked or coated in the liquid ‘urease’ test composition (see, for example, the above Table), then dried. This dried disc or ring, which carries all the necessary components of the urease test, will then suitably be inserted into any well system and suitably have a small volume of water added to dissolve the reagents in the disc/ring. The gastric material (for example a gastric biopsy fragment will then suitably be placed into the well to allow for the urease reaction to take place. The main advantages of this aspect of the invention include simplicity, rapid reaction and lower cost of blotter paper versus a tablet. The chief advantage of the ring versus a disc, is that when the gastric material is placed into the centre of the ring area, light comes through the ring hole allowing clear visibility (see, for example, FIG. 2).

FIG. 2 shows various views of a well system 10 in accordance with one embodiment suitable for receiving a coated or impregnated disc or ring in accordance with the present invention but which could also be used to receive a ringed tablet. Similarly to FIG. 1, the well system 10, suitably formed from a clear injection molded polypropylene, includes a well 2 to hold the disc or ring and an integrally hinged plug 3 to contain the solution in the well. The plug 3 is suitably optically clear to allow for viewing into the well system 10. The wet system 10 also includes a handle 4 which may include a frosted area to record a patients name. FIG. 2A shows the well system 10 in an open configuration and FIGS. 28 and 2C show the well system 10 in a dosed configuration. FIG. 2D shows a top view and FIG. 2E shows a cross-section along A-A of FIG. 2D. Dimensions of the well system 10 are shows in FIGS. 2D and 2E.

As shown in FIG. 2F, the well 2 includes a swaged rim 5 forming a ring shape and annular snap 6 so as to provide a liquid seal.

In use, as shown in FIG. 2F, a blot paper disc, in accordance with one embodiment of the present invention, having a centre hole and which is impregnated with the dry indicating composition is placed into the well 2 and retained by the swaged rim 5. Rehydration media, such as wafer (250 uL) is added into the well 2 so as to dissolve the dry indicating composition into solution. A biopsy 6 is placed into the well between the swaged rim 5 as shown and the solution allowed to react with any urease present, causing a colour change which can be viewed through plug 3.

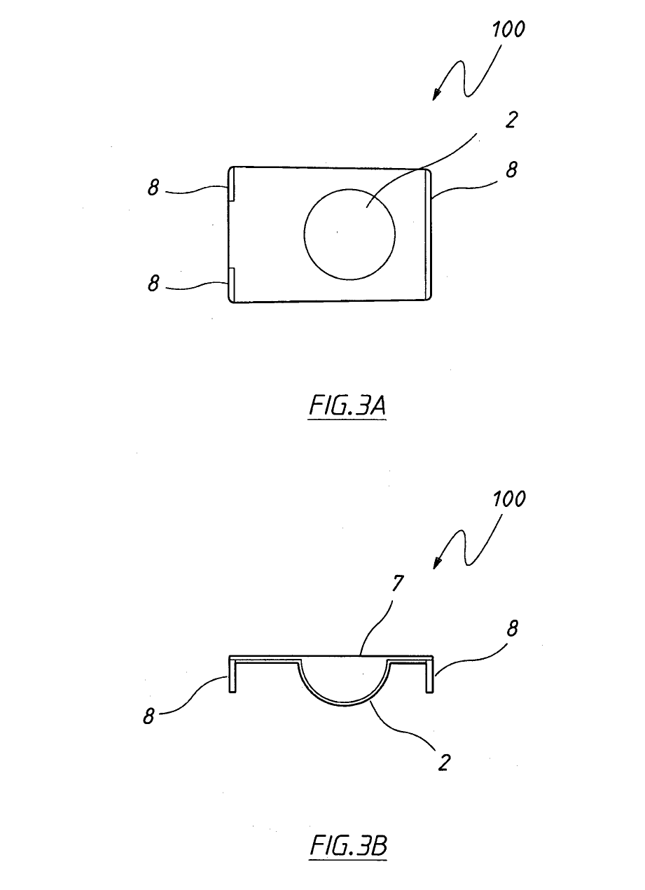

In a fourth aspect of this invention, the well is supplied to the physician pre-filled with liquid urease reagent such as described above in Table 1, but the well is sealed and presented in such a manner that it forms a stable platform. The physician performing the urease test simply peels back the seal, suitable a plasticized paper seal, inserts the gastric material (suitably a mucosa biopsy) and then reseals the test well (see FIG. 3). The colour change takes place and if the result is positive, the patient may be treated with anti-Helicobacter pylori antibiotic treatment. The unique feature is the airtight seal over the liquid urea reagent mix which markedly reduces the pH shift and prolongs the kit shelf-life by reducing the oxidation. The well may be manufactured in a nitrogen environment to further reduce oxygen contact with the urea reagent.

FIG. 3 shows an embodiment of a well system 100 in accordance with another embodiment. Similarly to FIG. 1, the well system 100 includes a solution well 2 for receiving a liquid urease indicating composition. In this embodiment however, the well 2 is sealed by an air-tight seal 7 (such as a sticker) which is peelable from the well and can be written on for patient identification. A thumb grip 8 may also be included. Suitably the seat is clear to allow viewing into the well 2.

A fifth aspect of the invention, is the form of the present invention where the complete composition of the urease test component, such as described above, is dissolved to form a liquid, then aliquoted into any well system, then dried. The urease chemicals are then suitably uniformly distributed around a well system ready for use as a reacting coating. Suitably on the day of endoscopic collection of gastric biopsies, several drops of water are suitably added to the well, so dissolving the dried coating of the urease test components and making the liquid ready for testing the biopsy. A gastric biopsy is then suitably inserted into the wall and the urease reaction then takes place. A clear advantage is the simplicity and low cost of the device. Another advantage is the status of the active agent prior to usage allows for a longer shelf life and the reagent remains system ready until activated. Prior to use the testing apparatus may be supplied in a closed configuration such that the testing apparatus remains sealed and therefore uncontaminated during storage and shipping.

The testing apparatus of the present invention is suitable for testing for the presence of urease, in order to diagnose gastrointestinal disorders of human or lower mammal subjects.

Testing is achieved by placing a sample of the gastric material, for example gastric mucosa (or gastric fluid), by means for example using tweezers (or forceps), against the dry indicating composition, and observing a color change in the dry indicating composition.

The observing step of this method suitably entails detection of any color change in the composition color, to the color exhibited by the indicator in its dissociation state. Failure of the composition to change color after about twenty-four hours reflects a negative test result.

The following non-limiting examples illustrate the compositions, methods and apparatus of the present invention.

### Example 1

The following example was performed in order to demonstrate the possibility of the urease testing tablet in accordance with the present invention.

A Stock Reagent solution of the above formulation (as described in Table above) was made up and kept under refrigeration to maintain stock solution integrity and tablets were created from this batch which were kept in dry state under room temperature conditions (23° C.) in a temperature controlled area. The stock solution was aliquoted into 4 mm plastic wells, allowed to dry then the same well was-realiqotted on 2 more occasions, dried and a flat ‘tablet’ removed 4 mm×1 mm A urease test well was prepared in the morning with a tablet inserted and a patient known to be as positive for Helicobacter pylori was tested. The urease test turned positive in 2 minutes. Another test using a tablet from the same batch of Stock reagent solution was tested six weeks later and the test was again repeated using a positive patient and a positive test was observed within 2.5 minutes. The shelf-life at approximately 23° C. of the test tablets was tested with urease powder and was able to remain unchanged visually with no signs of degradation or discolouration for seven months.

### Example 2

The following example was performed to demonstrate the possibility of urease testing using dried blotting paper containing the entire urease chemical composition.

A urease test was carried out using blotting paper which had been placed in the solution of the urease composition as described above in Table 1. The blotting paper was dried and a small, round disc of the blotting paper was cut out and placed in the well. Two drops of sterile water were added to the well. The solution allowed leaching process to take place and the necessary chemicals dissolved from the dried test paper to allow for a positive test to take place. Biopsy was taken from a patient with a duodenal ulcer. The urease test turned positive in 3 minutes.

### Example 3

The following example was performed to demonstrate utility of dried urease testing composition attached or coated to the wells for use in urease testing.

A series of urease test wells were prepared by placing two drops of a concentrated urease test solution (see Table 1 above) with and without a small amount of agar. These were dried over 24 hours. On the day of the endoscopic procedures, two drops of sterile water were placed in each well. Biopsies were taken from the antral mucosa from patients known to be positive for Helicobacter pylori. The urease test turned positive in between 3 and 12 minutes in the five as patients tested. The shelf-life of the dry product did not change for several months when stored in a temperature controlled area at 23° C.

A gelling agent may also be optionally incorporated in the initially wet indicating composition, preferably in the concentration of between about 5 to 50 grams per liter.

### Example 4

The following example was performed to demonstrate utility of the dried urease composition in filter paper cut as a ring and inserted into a specialized well which allows light penetration to heighten the visibility of the urease test. (FIG. 2).

Several specialty-designed urease wells were prepared with the dried filter paper rings inserted (FIG. 2). Three drops of water were dripped into the well system and after several minutes the urease solution was visible. Biopsies were then inserted into the center well. The reaction was clearly visible in 66 patients confirmed positive histologically requiring between 2 and 10 minutes to turn positive.

### Example 5

Several concentrations of the stock solution were produced in order to test the differences in intensity of reaction:

2× concentration of the above formulation was produced in a dried urease composition in filter paper, the filter paper was dried and cut into rings and inserted into a specialized well. The urease wells were then tested on three positive patients, two who were known positive and one patient undergoing screening procedures. The urease wells were activated using three drops of water and a two minutes were allowed to lapse to allow for reconstitution and reactivation of reagent Biopsy samples were added. A positive reaction was seen within 2 minutes for the two known positives and approximately 6 minutes for the remaining patient.

4× concentration of the above formulation was also produced in a dried urease composition in filter paper which was prepared as per the above method. The composition was tested on 5 patients three with known positive Helicobacter pylori infection on urea breath test, one recently treated with H. pylori eradication therapy and one known negative for H. pylori on urea breath test. Positive reaction times for the three positives were observed to range from 2-7 minutes and no positive reaction were observed in the remaining patients even after 24 hour observation period.

While the invention has been described with respect to a preferred embodiment, it will be understood that the invention is not limited to the preferred embodiment but is intended to cover various modifications and equivalent arrangements within the spirit and scope of the appended claims. This application is related to Australian Provisional Patent Application number 2010904631, the contents of which are incorporated herein by reference.

1. A method for detecting a urease in a gastric material taken from a mammal, comprising:
(a) providing a sample comprising a gastric material;
(b) providing a testing apparatus in the form of a tablet, a wafer, a coated or an impregnated material or a coated well,
wherein the tablet, wafer, coated or impregnated material or coated well comprises a dry indicating composition responsive to urease,
and the dry indicating composition comprises a urea and an indicating agent,
and the dry indicating composition is contacted with a water or an aqueous solution so as to hydrate the dry indicating composition or to bring the dry indicating composition into the aqueous solution prior to contacting with the gastric material;
(c) contacting the sample with the testing apparatus; and
(d) reading or determining a change in the color or detectable appearance of the rehydrated indicating composition, wherein a change in the color or detectable appearance indicates detection of the urease.

2. The method of claim 1, wherein the indicating composition is selected from the group consisting of a phenol red, a p-nitro-phenol, a bromothymol blue, a neutral red, a quinoline blue, a cresol red, a metacresol purple, a thymol blue, a bromocresol purple, a chlorophenol red, a bromocresol green and a bromophenol blue.

3. The method of claim 1, wherein the dry indicating composition is prepared by drying a stock solution comprising per 200 mL:
Urea between about 2 to 50 g
Indicating Agent between about 0.1 to 5.0 w/v (mg/L)

4. The method of claim 1, wherein in the testing apparatus, the urea has a mean particle size of less than 0.01 mm.

5. The method of claim 1, wherein the dry indicating composition further comprises one or more of. a disintegrant, a bacteriocidal agent, a preservative, a sodium dioxide, a sodium alumino silicate, a buffering agent, a pH adjusting agent, a solubilizing agent, a wetting agent, a diluent, a filler, a binder, a lubricant, a colorant, a stabilizer, a surfactant or an agar.

6. The method of claim 1, wherein a material of the coated or impregnated material or coated well comprises a filter paper or a blotting paper.

7. The method of claim 1, wherein the coated or impregnated material is in the form of a disc or ring.

8. The method of claim 1, wherein said tablet or wafer is prepared by compressing or molding the urea and the indicating composition to form the tablet or wafer.

9. The method of claim 1, wherein the testing apparatus is prepared by a method comprising immersing a material into a liquid comprising the urea and the indicating composition, followed by drying the liquid so as to form a coated or an impregnated material.

10. The method of claim 9, further comprising cutting the material into a disc or a ring.

11. The method of claim 1, wherein the testing apparatus is prepared by a method comprising coating a well with a liquid comprising a urea and an indicating agent, followed by drying the liquid so as to form a coated well.

12. A method for detection of a gastrointestinal disorder in a mammal by detection of a urease in a gastric material of the mammal, comprising the steps of:
(a) providing a testing apparatus in the form of a tablet, a wafer, a coated or an impregnated material or a coated well,
wherein the tablet, wafer, coated or impregnated material or coated well comprises a dry indicating composition responsive to urease,
and the dry indicating composition comprises a urea and an indicating agent,
and the dry indicating composition is contacted with a water or an aqueous solution so as to hydrate the dry indicating composition or to bring the dry indicating composition into the aqueous solution prior to contacting with the gastric material,
(b) obtaining a sample of a gastric material from said mammal;
(c) contacting the indicating composition with the gastric sample, and
(d) observing a colour change in the indicating composition,
wherein a change in colour in the indicating composition indicates the presence of a urease and the existence of a gastrointestinal disorder in the mammal.

13. The method of claim 12, wherein the contacting occurs in a well in which the tablet, wafer, coated or impregnated material has been placed, and the method further comprises adding water to hydrate the dry indicating composition or to bring the dry indicating composition into solution.

14. The method of claim 12, wherein the contacting occurs in the coated well, and the method further comprises adding water to hydrate the dry indicating composition or to bring the dry indicating composition into solution.

15. The method of claim 1, wherein the well forms part of a well system, or further comprises a well system comprising a well and an integrally hinged plug,
wherein the well comprises a swaged rim for retaining a disc or a ring inside the well, and the disc or ring comprises the dry indicating composition.

16. The method of claim 12, Wherein the well forms part of a well system, or further comprises a well system comprising a well and an integrally hinged plug,
wherein the well comprises a swaged rim for retaining a disc or a ring inside the well, and the disc or ring comprises the dry indicating composition.

17. The method of claim 12, wherein the gastrointestinal disorder comprises a disorder not manifested by presence of ulcerations in the gastric mucosa.

18. The method of claim 12, wherein the gastrointestinal disorder comprises a chronic or an atrophic gastritis, a gastroenteritis, a non-ulcer dyspepsia, an esophageal reflux disease, a gastric motility disorder, a peptic ulcer disease, a gastric ulcer or a duodenal ulcer.

19. The method of claim 12, wherein the gastrointestinal disorder comprises a disorder of the upper gastrointestinal tract caused or mediated by a bacteria, a Helicobacter-like organism or a Helicobacter pylori.

20. The method of claim 1, wherein the gastric material comprises a gastric epithelium, a gastric mucosa, a gastric tissue or a digestive fluid; or a biopsy sample, a vomitus sample or a nasal gastric aspirate sample; or a sample obtained from a biopsy, a vomitus or a nasal gastric aspirate.
